(19) United States
(12) Patent Application Publication
(10) Pub. No.: US 2010/0005570 A1
(43) Pub. Date: Jan. 14, 2010

(54) Disposable Sanitary Panty with System for Packaging and Marketing

(76) Inventor: Sharon-Or Rachman, Rishon Lezion (IL)

Correspondence Address: UZI EZRA HAVOSHA & PARTNERS, HADAR-DAFNA HOUSE 39 SHAOUL HAMELECH STREET, TEL AVIV, 64928, IL

(21) Appl. No.: 12/172,279

(22) Filed: Jul. 14, 2008

(51) Int. Cl. A41B 9/04; A61F 13/15

(52) U.S. Cl. 2/406; 604/385.14; 604/396

(57) ABSTRACT

A semi-disposable sanitary panty to be used by women comprising a supportive portion that can be wear more then one time and from an attached and removable portion includes pad with absorbent materials, that can be replaced and thrown after one time use. The semi-disposable sanitary panty is designed to answer the women need to be comfortable, feminine, and attractive, and still be disposable and thrown a way after being used one or more times.

The supportive portion is specially designed to fit the user's waist by hook members that are capable of being removably hooked onto each other for the purpose of opening and closing the supportive portion from at least one side of the supportive portion on the user's waist.

A system for marketing the semi-disposable sanitary panty is to sell packages were each package contains at least one supportive portion and few different pads that can be attached and after use removed and thrown away.

### FIELD OF THE INVENTION

This invention is in the field of underwear and in particular a semi-disposable sanitary panty with a system for packaging and marketing.

### BACKGROUND OF THE INVENTION

There are many patents in the field of female underwear probably because the requirements of women in this area are numerous. A partial list of such requirements is that the underwear be comfortable; attractive to look at; feeling of femininity; preferably not visible from the rear side when wearing tight fitting slacks; secure feeling that the various parts of the panty stay in place; an absorbent material to catch daily light discharges; the possibility of use for catching heavier discharges like the menstrual flow; the ability to change soiled parts with ease; conveniently removable without removing slacks; and disposable when no longer fresh.

This invention addresses these requirements to reveal an innovation in this field of female underwear.

### SUMMARY OF THE INVENTION

It is to be understood that both the foregoing general description and the following detailed description present embodiments of the invention and are intended to provide an overview or framework for understanding the nature and character of the invention as it is claimed.

This invention reveals a semi-disposable sanitary panty comprising a supportive portion that can be wear more than one time and an attachable hygienic portion that can be replaced after one time use.

The supportive portion circumscribes the region of the user's waist and has at least one break with a hook member attached to each side of the break which hook members that are capable of being removably hooked onto each other for the purpose of opening and closing the supportive portion. The supportive portion further includes a front portion and a rear portion. The front portion and the back portion attached to the supportive portion on the top side and are connected by a middle portion on the crotch area. The middle portion comprising a base made of non-moisture absorbing material and an absorbent sanitary pad. There might be release and attachment means to detach and attach this middle portion from and to the front and rear portions, so after one use the all middle part can be replaced.

The front portion of the semi-disposable sanitary panty is a triangular shaped attached to the supportive portion on its wider edge. The rear portion of the semi-disposable sanitary panty might be narrow like a string thereby creating a thong or string bikini effect. Alternatively the rear portion could comprise more material covering the buttocks than a thong or string bikini, known in the art as Brazilian style underwear or even more material covering the buttocks than a Brazilian style, known in the art as regular bikini underwear.

The attachment of the front portion and the rear portion to the middle portion might be with a secure attach and detach method like for example Velcro.

The hook members on the supportive portion might have adjustable positions to enable the panty to fit comfortably on different sized wearers.

The base of the middle portion is made of a material that prevents liquids passing through the material like for example a plastic or a rubber compound. Plastic could be of a type that is impervious to liquids but allows air to pass through as this is preferable for the health of the user. The sanitary pad is sufficiently absorbent to absorb light daily discharges. Alternatively the pad could be sufficiently absorbent to absorb a menstrual flow.

The supportive portion includes the front and back portion is made of one or more of the following materials suitable for underwear: cotton, viscose, and polyester. Spandex, elastane or lycra might be added to provide elasticize and stretchy effect.

The supportive portion is to be wear more then one time therefore the designed of the supportive portion would be such to give the woman who wears the semi-disposable sanitary panties to feel comfortable, feminine and attractive. Packaging for semi-disposable sanitary panties is also revealed in this invention. The panties could be packaged for sale with one or more supportive portions that may be used once or more than once according to the desire of the user. Together with the supportive portion could be packaged a number of middle portions with same or different thickness sanitary pads.

Alternatively the base of the middle portion might be as one unit with the supportive portion and only the absorbent sanitary pad can be removable attached to the base of the middle portion to be replaced after being used.

If the supportive portion of the semi-disposable sanitary panties made of one unit with the middle portion then a package might be sold with at least one supportive portion and with a number of sanitary pads to be attached to the supportive portion and be replaced after use. The sanitary pads might come in different sizes and thickness.

Another option is that the whole panty would come as one unit wherein the absorbent pad would be part of the middle portion that would be attached to the supportive portion. In such option the sanitary panties would be one time used and package might be sold with a number of panties with few different size and thickness of the sanitary pads.

### BRIEF DESCRIPTION OF THE DRAWINGS

The accompanying drawings are included to provide a further understanding of the invention and are incorporated into and constitute a part of this specification. The drawings illustrate various embodiments of the invention and, together with the description, serve to explain the principles and operations of the invention, by way of example only and not to limit the invention to these descriptions only.

FIG. 1 is a schematic depiction of a thong type semi-disposable sanitary panty in an open position.

FIG. 2 is a schematic depiction of a closed semi-disposable sanitary panty from the back view.

FIG. 3 is a schematic depiction of a closed semi-disposable sanitary panty from the front view and side view.

FIG. 4 is a schematic depiction of a semi-disposable sanitary panty in the style of a bikini.

FIG. 5 is a schematic depiction of a disposable sanitary panty in the style of a Brazilian bikini.

FIG. 6 is a schematic depiction of a disposable sanitary panty in the style of a thong bikini.

### DETAILED DESCRIPTION OF THE PREFERRED EMBODIMENTS

As will be appreciated the present invention is capable of other and different embodiments than those discussed above and described in more detail below, and its several details are capable of modifications in various aspects, all without departing from the spirit of the invention. Accordingly, the drawings and description of the embodiments set forth below are to be regarded as illustrative in nature and not restrictive.

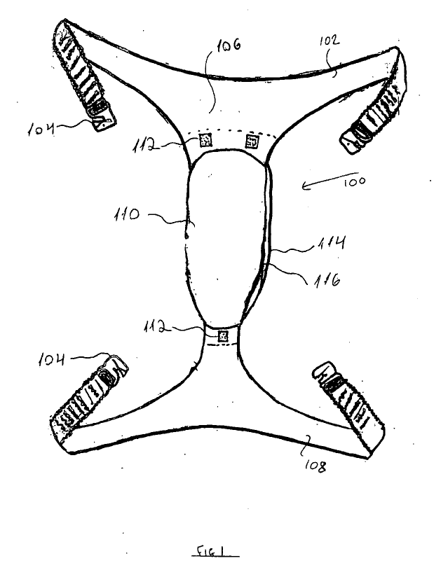

FIG. 1 shows an open position of semi-disposable sanitary panty 100 having a supportive portion 102 that circumscribes the region of the user's waist with at least one break with a hook member 104 attached to each side of the break. The hook members 104 are capable of being removably hooked onto each other for the purpose of opening and closing the supportive portion.

The supportive portion 102 further includes the front portion 106 and the rear portion 108 that are connected to the middle portion 110 that joins the front portion 106 and the rear portion 108 on the crotch area. Release and attachment means 112 are placed to detach and attach the middle portion 110 from and to the front portion 106 and rear portion 108, so after one use the all middle part 110 can be thrown away and be replaced by a new middle portion.

The middle portion comprising a base 114 made of non-moisture absorbing material that prevent liquids passing through the material like for example plastic or rubber compound, and an absorbent sanitary pad 116 to absorb light of heavier discharges.

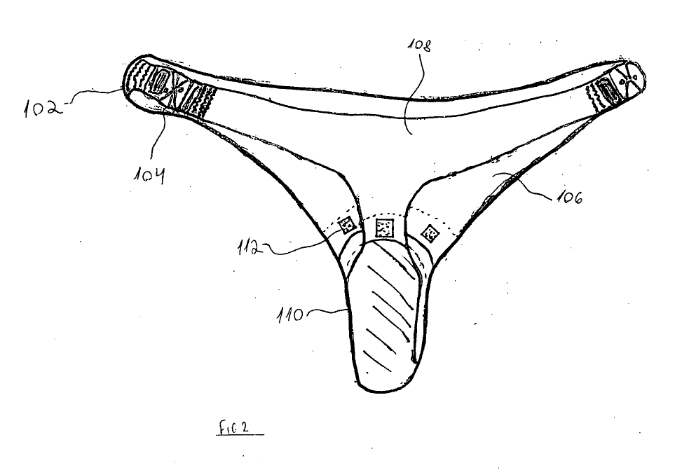

FIG. 2 shows a closed position of semi-disposable sanitary panty from the back view. The rear portion 108 and the front portion 106 are connected with release and attachment means 112 to the middle portion 110 on the crotch area.

The hook members 104 are hooked closing the supportive portion 102 to circumscribe the region of the user's waist, and are capable of being removable from each other for the purpose of opening the supportive portion 102, enabling the user to wear the semi-disposable sanitary panty with a grater comfort.

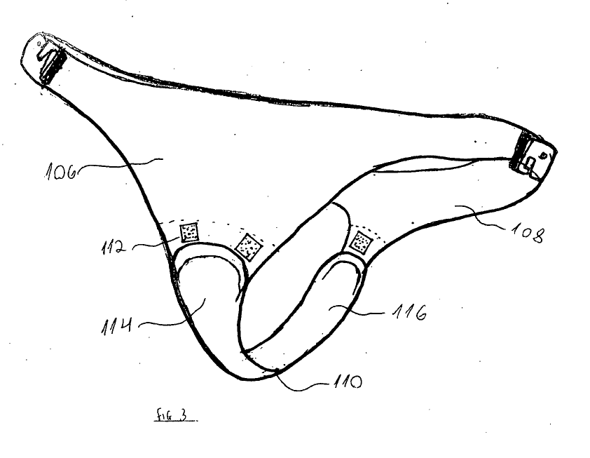

FIG. 3 shows a closed position of a semi-disposable sanitary panty from the front view and side view. The front portion 106 and the rear portion 108 are connected with release and attachment means 112 to the middle portion 110. The base 114 of the middle portion that made of non-moisture absorbing materials, is covering the external area of the semi-disposable sanitary panty and the sanitary pad 116 is covering the inward area of the middle portion 110.

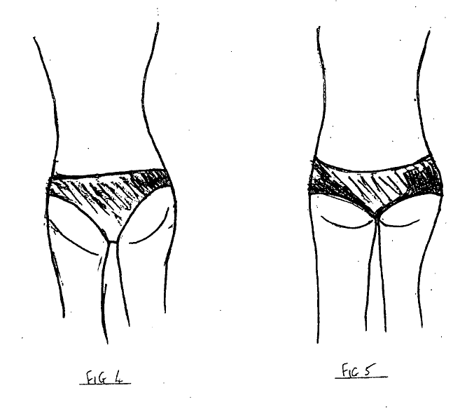

FIG. 4 shows a semi-disposable sanitary panty in the style of a bikini. The rear portion 106 made of a lot of material covering the buttocks known in the art as regular bikini underwear 120.

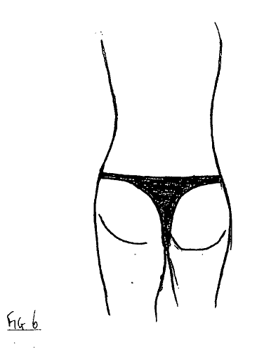

FIG. 5 shows a semi-disposable sanitary panty in the style of a Brazilian bikini. The rear portion made of less material, but still covering the buttocks known in the art as Brazilian style underwear 122.

FIG. 6 shows a semi-disposable sanitary panty in the style of a thong bikini. The rear portion made of very little material and is narrow like a string creating a thong or string bikini effect.

What is claimed:

1. A semi-disposable sanitary panty that comes in a package, said package includes
a. at least one supportive portion that circumscribes the region of the user's waist, said supportive portion comprising
i. substantially triangular shaped front portion wherein the wider edge of said front portion is attached to said supportive portion,
ii. a rear portion attached to said supportive portion,
iii. at least one break in said supportive portion with hook members attached to each side of the said break which hook members are capable of being removably hooked onto each other for the purpose of opening and closing the said supportive portion,
iv. release and attach means to detach and attach the middle portion from and to said supportive portion,
b. at least one middle portion, said middle portion comprising
i. a base made of materials that prevent liquids passing through material as plastic or rubber compound.
ii. an absorbent sanitary pad, including absorbent materials, attached on top of said base,
iii. release and attach means to detach and attach said middle portion from and to said supportive portion by connecting one side of said middle portion to said front portion and opposite side of said middle portion to said rear portions, so middle portion is placed in the crotch area, joining said front and rear portions.

2. A semi-disposable sanitary panty as claimed in claim 1 wherein said hook members have adjustable positions to enable said panty to fit comfortably on different sized wearers.

3. A semi-disposable sanitary panty as claimed in claim 1 wherein said rear portion is designed according to one of the following options:
a. said rear portion is narrow like a string thereby creating a thong or string bikini effect.
b. said rear portion comprises more material covering the buttocks than a thong or string bikini and is known in the art as Brazilian style underwear.
c. said rear portion comprises more material covering the buttocks than a Brazilian style and is known in the art as bikini underwear.

4. A semi-disposable sanitary panty as claimed in claim 1 wherein the attachment of said front and back portions to the middle portion is with a secure attach and detach method as Velcro.

5. A semi-disposable sanitary panty as claimed in claim 1 wherein said sanitary pad attached to said middle portion (bii) comply with one of the following options:
a. said sanitary pad is sufficiently absorbent to absorb light daily discharges.
b. said sanitary pad is sufficiently absorbent to absorb a menstrual flow.

6. Semi-disposable sanitary panty that comes in a package, said package includes
a. at least one supportive unit that circumscribes the region of the user's waist, said supportive portion comprising
i. substantially triangular shaped front portion wherein the wider edge of said front portion is attached to said supportive portion,
ii. a rear portion attached to said supportive portion,
iii. a middle portion attaches the front and rear portion on the crotch area, said middle portion made of materials that prevent liquids passing through, material as plastic or rubber compound,
iv. at least one break in said supportive portion with hook members attached to each side of the said break which hook members are capable of being removably hooked onto each other for the purpose of opening and closing the said supportive portion,
b. at least one sanitary pad made of absorbent material, said sanitary pad can be removably attached to said supportive portion on the said middle portion, and be replaced after being used.

7. A semi-disposable sanitary panty as claimed in claim 6 wherein said hook members have adjustable positions to enable said panty to fit comfortably on different sized wearers.

8. A semi-disposable sanitary panty as claimed in claim 6 wherein said rear portion is designed according to one of the following options:
a. said rear portion is narrow like a string thereby creating a thong or string bikini effect.
b. said rear portion comprises more material covering the buttocks than a thong or string bikini and is known in the art as Brazilian style underwear.
c. said rear portion comprises more material covering the buttocks than a Brazilian style and is known in the art as bikini underwear.

9. A semi-disposable sanitary panty as claimed in claim 6 wherein the attachment of said sanitary pad to said middle portion is with a secure attach and detach method as Velcro.

10. A semi-disposable sanitary panty as claimed in claim 6 wherein said sanitary pad (b) attached to said middle portion comply with one of the following options:
a. said sanitary pad is sufficiently absorbent to absorb light daily discharges.
b. said sanitary pad is sufficiently absorbent to absorb a menstrual flow.

11. A disposable sanitary panty made of one unit, comprising
a. A supportive portion circumscribes the region of the user's waist, includes the front rear and middle parts of the panty, said supportive portion further includes at least one break with hook members attached to each side of the said break which hook members are capable of being removably hooked onto each other for the purpose of opening and closing the said supportive portion. Said supportive portion is made of at least one of the following materials cotton, viscose, polyester, lycra, spandex, and elastance.
b. An upper layer, a sanitary pad like, including the absorbent material. Said upper layer attached on top of said supportive portion in the crotch area.
c. A lowest layer, said lowest layer made of materials that prevent liquids passing through as plastic or rubber compound. Said lowest layer is attached to the underneath of said supportive portion in the crotch area layer.

12. A semi-disposable sanitary panty as claimed in claim 11 wherein said hook members have adjustable positions to enable the said panty to fit comfortably on different sized wearers.

13. A semi-disposable sanitary panty as claimed in claim 11 wherein said supportive portion is made of very little materials, to be comfortable for wearing and be thrown away after one time use.

14. A semi-disposable sanitary panty as claimed in claim 11 wherein rear portion of said supportive portion is designed according to one of the following options:
a. said rear portion is narrow like a string thereby creating a thong or string bikini effect.
b. said rear portion comprises more material covering the buttocks than a thong or string bikini and is known in the art as Brazilian style underwear.
c. said rear portion comprises more material covering the buttocks than a Brazilian style and is known in the art as bikini underwear.

15. A semi-disposable sanitary panty as claimed in claim 11 wherein said upper lower of sanitary pad comply with one of the following:
a. said sanitary pad is sufficiently absorbent to absorb light daily discharges.
b. said sanitary pad is sufficiently absorbent to absorb a menstrual flow.
